(19) United States
(12) Patent Application Publication
(10) Pub. No.: US 2011/0202120 A1
(43) Pub. Date: Aug. 18, 2011

(54) Electrode Design for Reduced Trauma Insertion

(75) Inventors: Geoffrey R. Ball, Axams (AT); Martin Zimmerling, Patsch (AT)

(73) Assignee: MED-EL ELEKTROMEDIZINISCHE GERAETE GMBH, Innsbruck (AT)

(21) Appl. No.: 13/027,335

(22) Filed: Feb. 15, 2011

Related U.S. Application Data
(60) Provisional application No. 61/304,852, filed on Feb. 16, 2010.

(51) Int. Cl. A61N 1/05

(52) U.S. Cl. 607/137

(57) ABSTRACT

An implantable electrode for a cochlear implant system is described. A basal electrode lead goes from an implant housing to a cochleostomy opening and contains electrode wires for carrying one or more electrical stimulation signals. An apical electrode array fits through the cochleostomy opening into a patient cochlea and has multiple electrode contacts for applying the electrical stimulation signals to target neural tissue in the cochlea. Resilient array projections extend radially outward from an outer surface of the electrode array.

This application claims priority from U.S. Provisional Patent Application 61/304,852, filed Feb. 16, 2010, incorporated herein by reference.

### FIELD OF THE INVENTION

The present invention relates to medical implants, and more specifically to cochlear implant systems.

### BACKGROUND ART

A normal ear transmits sounds as shown in FIG. 1 through the outer ear 101 to the tympanic membrane (eardrum) 102, which moves the bones of the middle ear 103, which in turn vibrate the oval window and round window openings of the cochlea 104. The cochlea 104 is a long narrow duct wound spirally about its axis for approximately two and a half turns. The cochlea 104 includes an upper channel known as the scala vestibuli and a lower channel known as the scala tympani, which are connected by the cochlear duct. The scala tympani forms an upright spiraling cone with a center called the modiolar where the spiral ganglion cells of the acoustic nerve 113 reside. In response to received sounds transmitted by the middle ear 103, the fluid filled cochlea 104 functions as a transducer to generate electric pulses that are transmitted to the cochlear nerve 113, and ultimately to the brain. Hearing is impaired when there are problems in the ability to transduce external sounds into meaningful action potentials along the neural substrate of the cochlea 104.

In some cases, hearing impairment can be addressed by a cochlear implant that electrically stimulates auditory nerve tissue with small currents delivered by multiple electrode contacts distributed along an implant electrode. FIG. 1 shows some components of a typical cochlear implant system where an external microphone provides an audio signal input to an external signal processing stage 111 which implements one of various known signal processing schemes. The processed signal is converted by the external signal processing stage 111 into a digital data format, such as a sequence of data frames, for transmission into a receiver processor in an implant housing 108. Besides extracting the audio information, the receiver processor in the implant housing 108 may perform additional signal processing such as error correction, pulse formation, etc., and produces a stimulation pattern (based on the extracted audio information) that is sent through wires in an electrode lead 109 to an implanted electrode array 110. Typically, the electrode array 110 includes multiple electrodes on its surface that provide selective stimulation of the cochlea 104.

The electrode array 110 penetrates into the cochlea 104 through a surgical opening called a cochleostomy. The electrode array 110 has multiple electrode contacts on or slightly recessed below its outer surface for applying one or more electrical stimulation signals to target audio neural tissue within the cochlea 104. The extra-cochlear electrode lad 109 that goes from the implant housing 108 to the cochleostomy opening usually has no electrical contacts except perhaps a ground electrode and it encloses connecting wires that deliver electrical stimulation signals to the electrode contacts on the electrode array 110.

Insertion and placement and insertion of the electrode array 110 into the cochlea 104 causes trauma to the cochlear tissue due to the rigidity, friction, and impact of moving the electrode array 110 through the cochlea 104. For example, insertion of the electrode array 110 may damage soft tissues, membranes, thin bony shelves, blood vessels, neural elements, etc. In the case of multiple insertions, the damage can accumulate. In addition, removal and replacement of the electrode array 110 due to device failure or aging is also a serious problem. For example, patients with some residual hearing now receive hybrid implant systems that also include acoustic-mechanical stimulation components, and further hearing loss could occur when the electrode array 110 is removed or replaced. In addition, there are efforts to use therapeutic drugs to regrow neural tissue around an inserted electrode array 110 which could suffer catastrophic consequences when the electrode is removed since any new neural tissue growth that might reach the electrode could be disrupted or destroyed.

Thus, designers of the electrode array 110 work hard to ensure that it is soft and flexible to minimize the insertion trauma. The electrode array 110 also is constrained to have a uniform external aspect with a smooth outer surface. The impact of electrode insertion in certain regions of the inner ear is also addressed by using a pre-shaped (i.e., pre-curved) electrode array 110. But the issues associated with cummulative permanent trauma due to multiple explantation and re-implantion of the electrode array 110 has not been addressed.

U.S. Pat. No. 5,922,017 shows an example of a cochlear implant electrode in FIG. 3a that has an irregular-shape section in the middle which may have some utility in the insertion process, but there is no discussion provided for this feature.

### SUMMARY OF THE INVENTION

Embodiments of the present invention are directed to an implantable electrode for a cochlear implant system that minimizes trauma when inserted. A basal electrode lead goes from an implant housing to a cochleostomy opening and contains electrode wires for carrying one or more electrical stimulation signals. An apical electrode array fits through the cochleostomy opening into a patient cochlea and has multiple electrode contacts for applying the electrical stimulation signals to target neural tissue in the cochlea. Resilient array projections extend radially outward from an outer surface of the electrode array.

In some specific embodiments, the array projections may be arranged in a parallel planes each containing multiple projections. For example, each plane may contain three equidistant array projections. The array projections include may include angled pointed barb projections. The array projections may have a height of between 10 μm and 500 μm, for example, less than 100 μm. The array projections may be biologically resorbable over time into surrounding tissue. The array projections may include a lubricant coating, an anti-inflammatory coating, and/or a therapeutic pharmaceutical coating.

### BRIEF DESCRIPTION OF THE DRAWINGS

FIG. 1 shows elements of a human ear having a typical cochlear implant system.

FIG. 2 shows features of an implantable electrode according to one embodiment of the present invention.

FIG. 3 A-B illustrates how the insertion projections operate on the middle electrode section of an embodiment of the present invention.

FIG. 4 A-D shows an embodiment of an implantable electrode having array projections on the electrode array.

FIG. 5 A-B shows another embodiment of an electrode array having micro-projections on the electrode array.

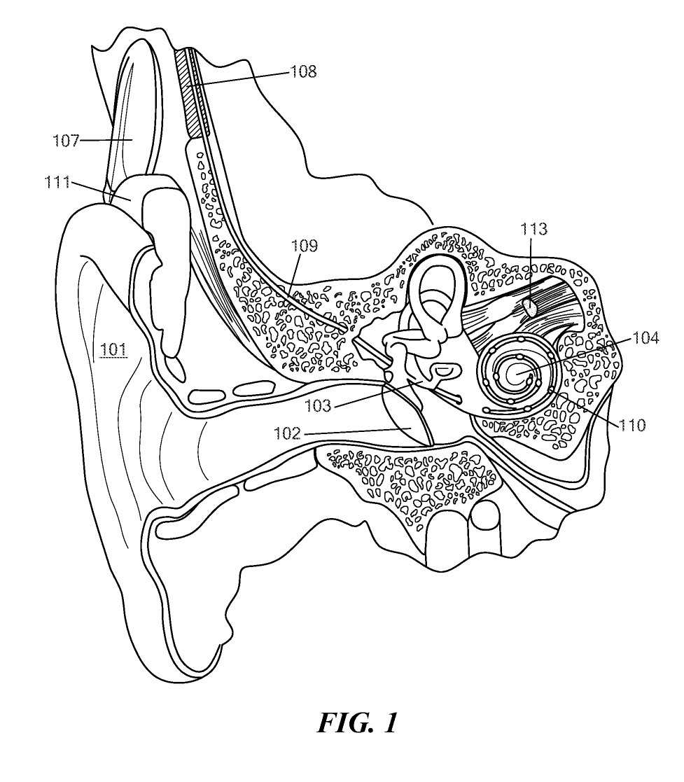

### DETAILED DESCRIPTION OF SPECIFIC EMBODIMENTS

As explained above, it is highly desirable to minimize trauma to the adjacent tissues when inserting a cochlear implant electrode. Embodiments of the present invention are directed to an implantable electrode for a cochlear implant system that minimizes trauma when inserted by making it easier to insert the electrode to the optimal depth in the cochlea while minimizing back and forth movement, and then immobilizing the electrode in that position.

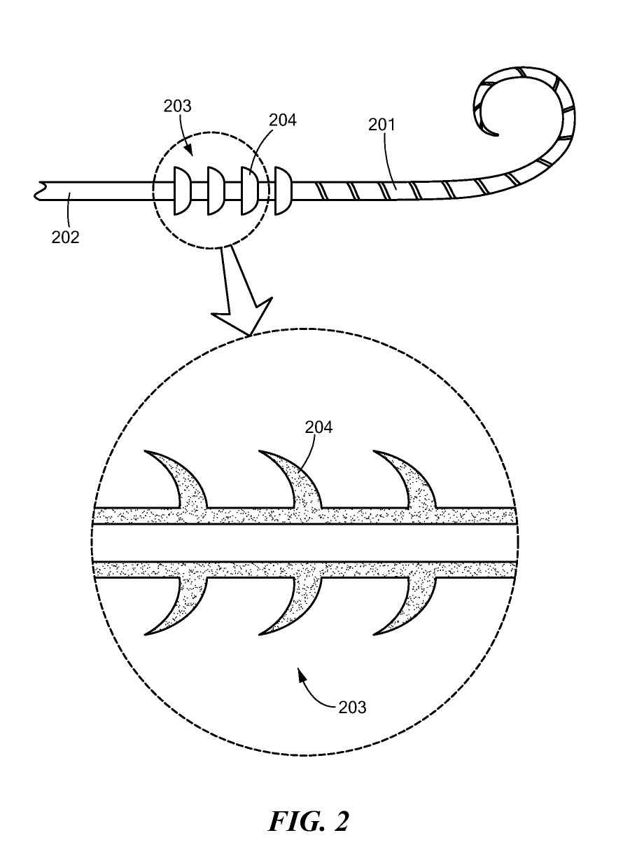

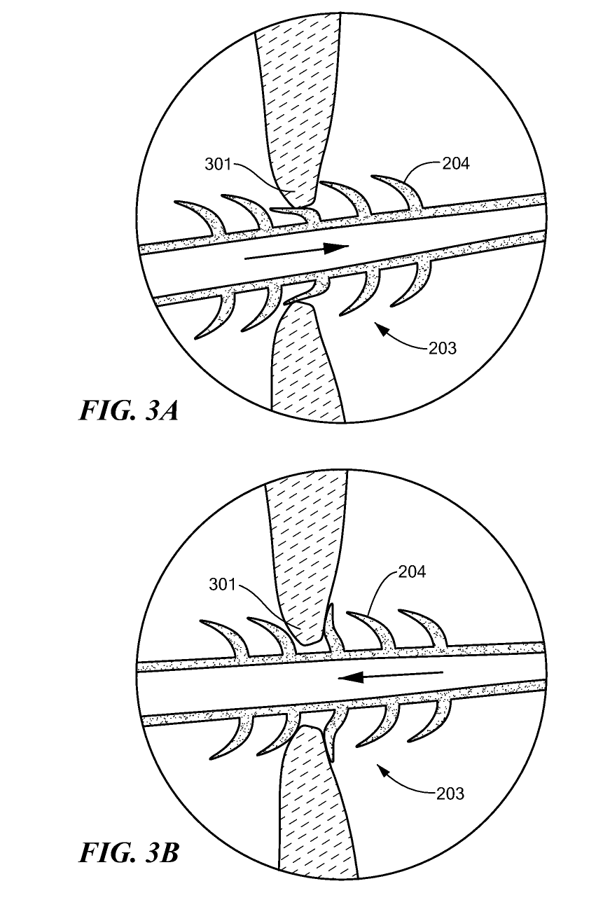

FIG. 2 shows features of an implantable electrode according to one embodiment of the present invention having a basal electrode lead 202 that passes from an implant housing to a cochleostomy opening for carrying one or more electrical stimulation signals from the implant housing. The cochleostomy opening may be through the round window membrane, the oval window membrane, the promontory, or the apical turn of the cochlea. An apical electrode array 201 fits through the cochleostomy opening into a cochlea and has electrode contacts for applying the electrical stimulation signals to target neural tissue in the cochlea. A cylindrical middle electrode section 203 connects the electrode lead 202 and the electrode array 201 and includes an outer surface having angled resilient projections 204. In some specific embodiments, the resilient projections 204 may be angled circular flange projections and/or angled pointed barb projections. And the electrode array 201 may include a lubricant coating, an anti-inflammatory coating and/or a therapeutic pharmaceutical coating.

FIG. 3 illustrates how the resilient projections 204 operate on the middle electrode section 203 of an embodiment of the present invention. In FIG. 3A, as the middle electrode section 203 is pushed through the cochleostomy opening 301, the resilient projections 204 are compressed. Because the resilient projections 204 are angled backward, this arrangement provides for smooth passage of the middle electrode section 203 through the cochleostomy opening 301 when inserting the electrode array 201 into the cochlea. And, as shown in FIG. 3B, the backwards angle of the resilient projections 204 resists withdrawal of the middle electrode section 203 from the cochleostomy opening 301 and maintains it at the correct depth of insertion without further movement.

In some specific embodiments, the middle electrode section 203 may include a color coding and/or number coding arrangement to indicate to the surgeon insertion depth of the electrode array 201 into the cochlea. Thus, pre-surgical imaging such as magnetic resonance imaging (MRI) may be used to determine the exact size, shape and position of the patient's cochlea, and from that, the surgeon may calculate exactly how far into the cochlea to insert the electrode array 201 for optimal post-surgical operation. Then the resilient projections 204 together with any position coding arrangements such as color or number indexing may be used to help the surgeon determine when the electrode array 201 has been correctly inserted to the nominal pre-determined depth. By helping the surgeon to correctly introduce the electrode array 201 into the cochlea with minimal back and forth movement helps minimize trauma to the cochlear tissues from the introduction of the electrode. And the resistance of the resilient projections 204 to withdrawal from the cochleostomy opening 301 helps ensure that the electrode stays in correct position after surgery, further reducing post-surgical trauma and degradation of the implant system.

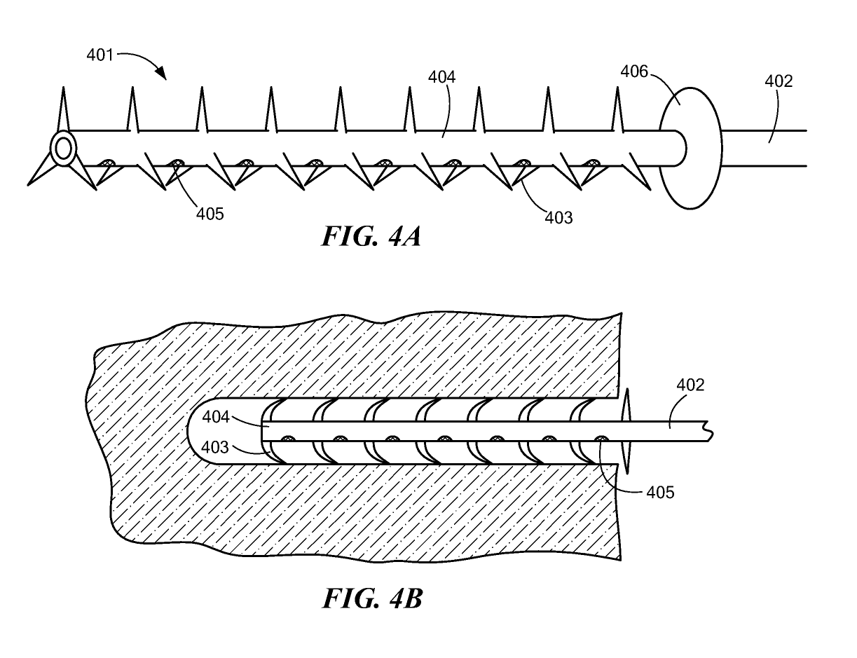

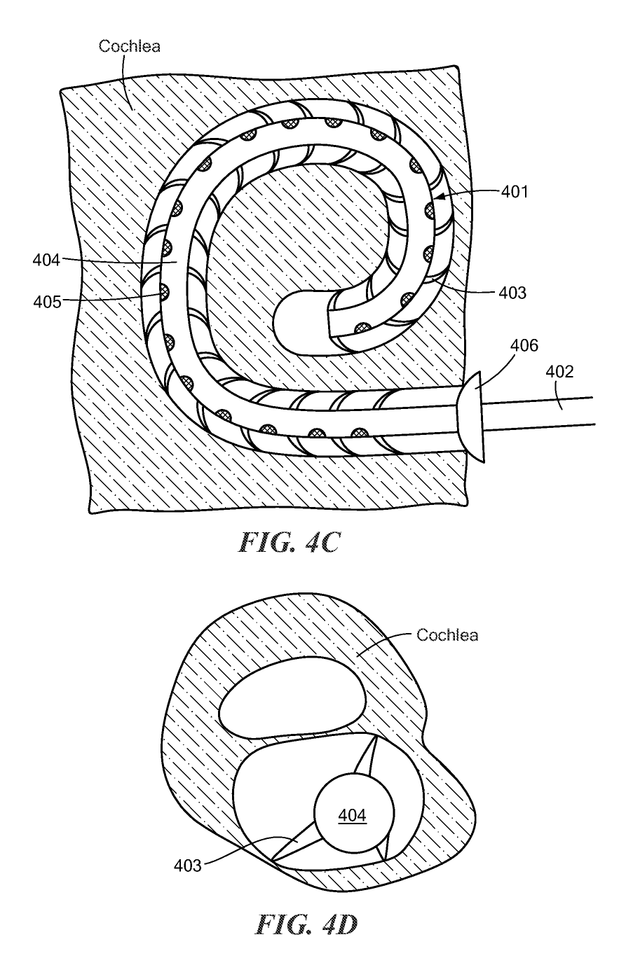

FIG. 4 A-D shows an embodiment of an electrode having array projections 403 extending radially outward from an outer surface on the electrode array 401. In the embodiment shown in FIG. 4A, the array projections 403 are arranged in a parallel planes each containing three equidistant projections. In some such embodiments, the array projections 403 may be small diameter angled pointed barb projections having soft silicone elastomer tips that bend back as the electrode array 401 is inserted into target tissue, as shown for example, in FIG. 4B. FIG. 4C is a side view and FIG. 4D a cross-section view showing how such very soft array projections 403 can help align the electrode array 401 as it is inserted within the cochlear scala while minimizing the surface area of the electrode array 401 that contacts the delicate cochlear tissue structures. This in turn may reduce the required insertion force, and reduce insertion trauma and maximize the preservation of residual hearing in the patient.

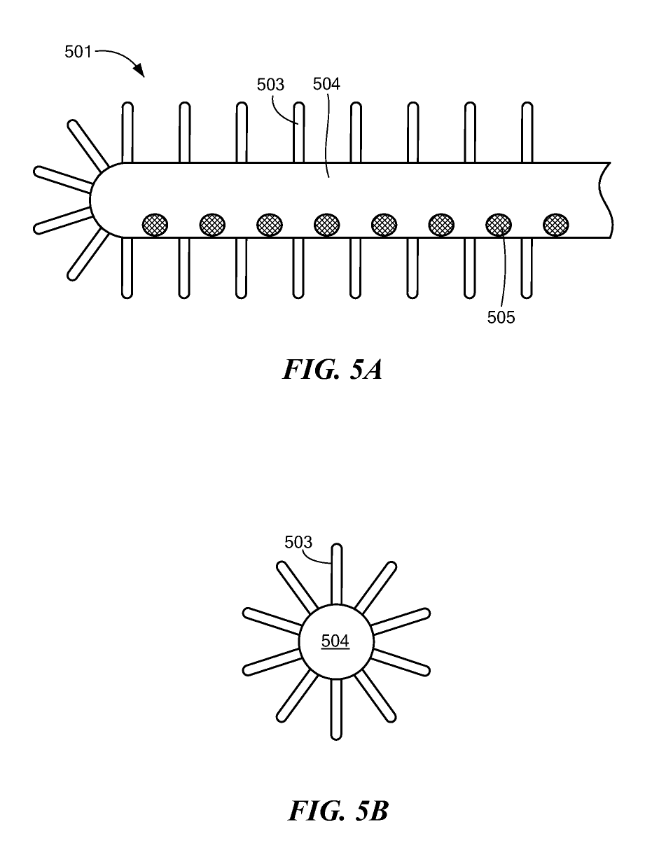

FIG. 5A is a side view and FIG. 5B a cross-section view showing another embodiment of an electrode array 501 having radial micro-projections 503 with a height of between 10 μm and 500 μm, for example, less than 100 μm. The array projections 503 may be biologically resorbable over time into surrounding tissue. And in some embodiments, the array projections 503 may include a lubricant coating, an anti-inflammatory coating, and/or a therapeutic pharmaceutical coating.

Although various exemplary embodiments of the invention have been disclosed, it should be apparent to those skilled in the art that various changes and modifications can be made which will achieve some of the advantages of the invention without departing from the true scope of the invention.

What is claimed is:

1. An implantable electrode for a cochlear implant system comprising:
a basal electrode lead from an implant housing to a cochleostomy opening containing a plurality of electrode wires for carrying one or more electrical stimulation signals;
an apical electrode array fitting through the cochleostomy opening into a patient cochlea and having a plurality of electrode contacts for applying the electrical stimulation signals to target neural tissue in the cochlea; and
a plurality of resilient array projections extending radially outward from an outer surface of the electrode array.

2. An implantable electrode according to claim 1, wherein the array projections are arranged in a plurality of parallel planes, each plane having a plurality of projections.

3. An implantable electrode according to claim 2, wherein each plane contains three equidistant array projections.

4. An implantable electrode according to claim 1, wherein the array projections include angled pointed barb projections.

5. An implantable electrode according to claim 1, wherein the array projections have a height of between 10 μm and 500 μm

6. An implantable electrode according to claim 5, wherein the array projections have a height of less than 100 μm.

7. An implantable electrode according to claim 1, wherein the array projections are biologically resorbable over time into surrounding tissue.

8. An implantable electrode according to claim 1, wherein the array projections include a lubricant coating.

9. An implantable electrode according to claim 1, wherein the array projections include an anti-inflammatory coating.

10. An implantable electrode according to claim 1, wherein the array projections include a therapeutic pharmaceutical coating.
